(19) United States
(12) Patent Application Publication
(10) Pub. No.: US 2025/0288226 A1
(43) Pub. Date: Sep. 18, 2025

(54) METHOD AND APPARATUS FOR IMPROVING LAG CORRECTION DURING IN VIVO MEASUREMENT OF ANALYTE CONCENTRATION WITH ANALYTE CONCENTRATION VARIABILITY AND RANGE DATA

(71) Applicant: ABBOTT DIABETES CARE INC., Alameda, CA (US)

(72) Inventor: Erwin Satrya Budiman, Fremont, CA (US)

(73) Assignee: ABBOTT DIABETES CARE INC., Alameda, CA (US)

(21) Appl. No.: 19/221,831

(22) Filed: May 29, 2025

Related U.S. Application Data
(63) Continuation of application No. 18/438,948, filed on Feb. 12, 2024, now Pat. No. 12,343,140.
Application No. 18/438,948 is a continuation of application No. 17/073,852, filed on Oct. 19, 2020, now Pat. No. 11,896,371.
Application No. 17/073,852 is a continuation of application No. 15/910,927, filed on Mar. 2, 2018, now Pat. No. 10,842,420.
Application No. 15/910,927 is a continuation of application No. 14/431,168, filed on Mar. 25, 2015, now Pat. No. 9,907,492.
(60) Provisional application No. 61/705,929, filed on Sep. 26, 2012.

(51) Int. Cl. A61B 5/145; A61B 5/00; G16H 50/20

(52) U.S. Cl. CPC A61B 5/14532; A61B 5/14503; A61B 5/14546; A61B 5/7203; A61B 5/7235; A61B 5/7275; G16H 50/20; A61B 2560/0475; G06F 2218/10

(57) ABSTRACT

Methods, devices, and systems are provided. A method includes receiving sensor data from the glucose sensor, the sensor data being associated with a time window, determining a rate of change distribution of the sensor data, determining a first slope that corresponds to a line of the rate of change distribution, comparing a first slope with a first reference slope, generating, based on comparing the first slope with the first reference slope, an output; and, transmitting the output to a display or a transmitter. Numerous additional features are disclosed.

### PRIORITY

The present application is a continuation of U.S. patent application Ser. No. 18/438,948 filed Feb. 12, 2024, which is a continuation of U.S. patent application Ser. No. 17/073,852 filed Oct. 19, 2020, now U.S. Pat. No. 11,896,371, which is a continuation of U.S. patent application Ser. No. 15/910,927 filed Mar. 2, 2018, now U.S. Pat. No. 10,842,420, which is a continuation of U.S. patent application Ser. No. 14/431,168 filed Mar. 25, 2015, now U.S. Pat. No. 9,907,492, which is a national stage patent application under 35 U.S.C. § 371, which claims priority to PCT Application No. PCT/US13/60471 filed Sep. 18, 2013, which claims priority to U.S. Provisional Application No. 61/705,929 filed Sep. 26, 2012, entitled “Method and Apparatus for Improving Lag Correction During In Vivo Measurement of Analyte Concentration with Analyte Concentration Variability and Range Data”, the disclosures of each and every one of which are incorporated herein by reference in their entirety for all purposes.

### BACKGROUND

The detection of the concentration level of glucose or other analytes in certain individuals may be vitally important to their health. For example, the monitoring of glucose levels is particularly important to individuals with diabetes or pre-diabetes. People with diabetes may need to monitor their glucose levels to determine when medication (e.g., insulin) is needed to reduce their glucose levels or when additional glucose is needed.

Devices have been developed for automated in vivo monitoring of analyte concentrations, such as glucose levels, in bodily fluids such as in the blood stream or in interstitial fluid. Some of these analyte level measuring devices are configured so that at least a portion of the devices are positioned below a skin surface of a user, e.g., in a blood vessel or in the subcutaneous tissue of a user. As used herein, the term analyte monitoring system is used to refer to any type of in vivo monitoring system that uses a sensor disposed with at least a portion subcutaneously to measure and store sensor data representative of analyte concentration levels automatically over time. Analyte monitoring systems include both (1) systems such as continuous glucose monitors (CGMs) which transmit sensor data continuously or at regular time intervals (e.g., once per minute) to a processor/display unit and (2) systems that transfer stored sensor data in one or more batches in response to a request from a processor/display unit (e.g., based on an activation action and/or proximity, for example, using a near field communications protocol) or at a predetermined but irregular time interval.

Determining an analyte concentration level in blood based on the analyte concentration in interstitial fluid can be difficult because changes of the analyte concentration levels in interstitial fluid typically lags behind changing analyte concentration levels in blood. Thus, what is needed are systems, methods, and apparatus to correct for the time lag between blood analyte level changes and interstitial fluid analyte level changes.

### SUMMARY

Methods, devices, and systems are provided for correcting time lag in measurements of analyte concentration level in interstitial fluid. When applied to lag correction of glucose using analyte monitoring system (e.g., CGM) sensor data measuring glucose in interstitial fluid, the degree of glycemic variability and/or range are used to determine the relative benefit of relying on the computed glucose rate of change for lag correction versus the risk of reduced precision caused by amplifying noise and other artifacts. Thus, in some embodiments, the invention includes determining the analyte concentration variability of a patient and/or the analyte concentration range of a patient and determining a lag correction value to apply to sensor data representative of analyte concentration measured in interstitial fluid using an analyte measurement system. The lag correction value is adjusted based upon the analyte concentration variability and/or analyte concentration range. Finally, an analyte concentration level representative of the blood analyte concentration level is computed based on the adjusted lag correction value. Related systems and computer program products are also disclosed.

In some embodiments, the invention includes receiving a signal representative of sensor data from an analyte monitoring system related to an analyte level of a patient measured over time. Rates of change of the sensor data for a time period of the sensor data are computed along with a rate distribution of the rates of change. The rate distribution is transformed into a linear arrangement, a best-fit line is determined for the transformed rate distribution, a slope of the best-fit line is computed, and a scaling factor for lag correction is determined. The slope of the best-fit line is used as a representation of the variability of the analyte level to adjust an amount of lag correction applied to the sensor data by adjusting the scaling factor for lag correction. Related systems and computer program products are also disclosed.

Some other embodiments of the present disclosure include computer-implemented methods of correcting lag in measurements of analyte concentration level in interstitial fluid. The methods include defining a scaling factor for lag correction, collecting a moving window of historical analyte sensor data, defining a probability density function of the sensor data within the moving window, determining a normalized analyte variability ratio, storing the normalized analyte variability ratio computed at regular intervals, comparing a latest normalized analyte variability ratio to a predetermined value and a number of prior values, setting a value of the scaling factor based on the probability density function, and computing lag corrected values based on the scaling factor. Related systems and computer program products are also disclosed.

Yet other embodiments of the present disclosure include additional and alternative methods of correcting lag in measurements of analyte concentration level in interstitial fluid. The methods include determining at least one of analyte concentration variability of a patient and analyte concentration range, determining a lag correction value to apply to sensor data representative of analyte concentration measured in interstitial fluid using an analyte measurement system, adjusting the lag correction value based upon the at least one of analyte concentration variability and analyte concentration range, and computing an analyte concentration level representative of a blood analyte concentration level based on the adjusted lag correction value. Related systems and computer program products are also disclosed.

Numerous other aspects and embodiments are provided. Other features and aspects of the present invention will become more fully apparent from the following detailed description, the appended claims, and the accompanying drawings.

### BRIEF DESCRIPTION OF THE DRAWINGS

FIG. 1 depicts a plot of an example analyte concentration rate of change distribution in accordance with some embodiments of the present invention.

FIG. 2 depicts a plot of an example transformed analyte concentration rate of change distribution in accordance with some embodiments of the present invention.

FIG. 3 depicts a plot of example best-fit lines of a transformed analyte concentration rate of change distribution in accordance with some embodiments of the present invention.

FIG. 4 depicts a flowchart illustrating an example of a method of determining glucose variability in accordance with some embodiments of the present invention.

FIG. 5 depicts a flowchart illustrating an example of a method of lag correction based on glucose variability in accordance with some embodiments of the present invention.

FIG. 6 depicts a flowchart illustrating an example of a method of monitoring glycemic control based on glucose variability in accordance with some embodiments of the present invention.

FIGS. 7A to 7C depict plots of example glucose levels over time, corresponding rate of change of the glucose levels over time, and best-fit lines of the corresponding transformed glucose concentration rate of change distribution, respectively and in accordance with some embodiments of the present invention.

FIG. 8 depicts a flowchart illustrating an example of a method of lag correction based on glucose range in accordance with some embodiments of the present invention.

### DETAILED DESCRIPTION

The present invention provides systems, methods, and apparatus to improve lag correction in devices that determine analyte concentration in the blood via measurement of the analyte concentration in interstitial fluid. For such devices, determining blood glucose levels, for example, may involve performing lag correction based on a calculated estimate of rates of change of blood glucose levels. However, the accuracy of computing the rates of change can be very sensitive to noise. It has been observed that in patients with relatively good glycemic control (i.e., relatively low blood glucose level variability), the relative performance improvement due to lag correction is not as significant as in subjects with poorer control (i.e., relatively high blood glucose level variability). In some cases, the risk of reduced accuracy due to rate calculation error increases because a higher fraction of the computed rate is due to noise and other artifacts.

Improving lag correction is thus a tradeoff between maximal smoothing (i.e., increasing precision) during periods of noisy, unchanging levels and maximal lag correction (i.e., increasing accuracy) during periods of non-noisy, rapidly changing levels. Therefore, given a constant noise level, a relatively unchanging glucose level benefits from less lag correction than a relatively rapidly changing glucose level. Existing methods of lag correction may rely on estimating the glucose level trend and minute-by-minute noise level to determine the amount of smoothing to apply. In contrast, the present invention uses information beyond the time span in which the signals are still highly correlated, to get a more global sense of the patient's glucose level variability.

In some embodiments, the present invention considers rates of change of glucose concentration levels based on glucose measurements over time and assesses the degree of glucose level variability that is relatively insensitive to noise and other artifacts. The degree of glucose level variability is usable in several ways. In some embodiments, the degree of glucose level variability is used to help determine the amount of tradeoff between maximizing lag correction of interstitial glucose measurements and minimizing output noise. In some embodiments, the degree of glucose level variability is being used to aid in measuring a patient's degree of glycemic control.

In addition to considering the rate of change of glucose levels, considering the range of a patient's glucose levels can also be used to improve lag correction according to the present invention. The factors that reduce precision affect lag correction more at the extreme ends of a patient's glucose excursion. For example, at the lower end of a patient's glucose levels, the levels can be affected by dropouts and other signal artifacts in a higher percentage than at the higher end. In other words, a 30 mg/dL dropout at a 60 mg/dL glucose level is a 50% error while the same 30 mg/dL dropout at a 180 mg/dL level is only a 17% error. As a result, the risk of introducing error when lag correcting to the full extent differs in these different glucose level ranges. Thus, considering the range of a patient's glucose levels and the patient's level patterns can be used to relate the risk of making a lag correction and the factors that reduce precision.

Since a patient's glucose levels do not regularly follow a repetitive pattern and patients have different patterns that can change over time, a static plot of a patient's glucose response to a meal, for example, is not likely to be useful for gauging the range of a patient's glucose levels. However, by starting with conservative nominal values and storing glucose variability and excursion range statistics computed from measurements taken over a period of time (e.g., a window of hours or days), a more accurate characterization of the patient's changing glucose range can be determined. Using this slowly changing range, the relative position of the most recently measured glucose level compared to the patient's history can be determined. When the most recently measured glucose value is in the lower range of the patient's historic range, then the amount of lag correction applied can be reduced by a predetermined amount as a function of the most recently measured glucose value and one or more slowly changing statistics collected from historical sensor data of the patient. When the most recently measured glucose value is in the middle range of the patient's historic range, the amount of lag correction applied can be set to the maximum. At the higher range of the historic range, the amount of lag correction can be reduced as with the lower range. Thus, in this manner, the amount of lag correction can be reduced at the extremes of the patient's glucose excursions.

Embodiments of the invention are described primarily with respect to continuous glucose monitoring devices and systems but the present invention can be applied to other analytes, other analyte characteristics, and other analyte measurement systems, as well as data from measurement systems that transmit sensor data from a sensor unit to another unit such as a processing or display unit in response to a request from the other unit. For example, other analytes that can be monitored include, but are not limited to, acetyl choline, amylase, bilirubin, cholesterol, chorionic gonadotropin, creatine kinase (e.g., CK-MB), creatine, DNA, fructosamine, glutamine, growth hormones, hormones, ketones, lactate, peroxide, prostate-specific antigen, prothrombin, RNA, thyroid stimulating hormone, and troponin. The concentration of drugs, such as, for example, antibiotics (e.g., gentamicin, vancomycin, and the like), digitoxin, digoxin, drugs of abuse, theophylline, and warfarin, can also be monitored. In those embodiments that monitor more than one analyte, the analytes can be monitored at the same or different times. In addition, in some embodiments, the present invention can be applied to non-analyte sensor data. For example, non-analyte sensor data can include temperature estimation of a target physiological compartment that is made based on measuring the temperature of a nearby compartment, where the measured temperature lags from the temperature of the target compartment. The present invention also provides numerous additional embodiments.

Some embodiments of the present invention include a programmed computer system adapted to receive and store data from an analyte monitoring system. The computer system can include one or more processors for executing instructions or programs that implement the methods described herein. The computer system can include memory and persistent storage devices to store and manipulate the instructions and sensor data received from the analyte monitoring system. The computer system can also include communications facilities (e.g., wireless and/or wired) to enable transfer of the sensor data from the analyte monitoring system to the computer. The computer system can include a display and/or output devices for identifying dropouts in the sensor data to a user. The computer system can include input devices and various other components (e.g., power supply, operating system, clock, etc.) that are typically found in a conventional computer system. In some embodiments, the computer system is integral to the analyte monitoring system. For example, the computer system can be embodied as a handheld or portable receiver unit within the analyte monitoring system.

In some embodiments, the various methods described herein for performing one or more processes, also described herein, can be embodied as computer programs (e.g., computer executable instructions and data structures). These programs can be developed using an object oriented programming language, for example, that allows the modeling of complex systems with modular objects to create abstractions that are representative of real world, physical objects and their interrelationships. However, any practicable programming language and/or techniques can be used. The software for performing the inventive processes, which can be stored in a memory or storage device of the computer system described herein, can be developed by a person of ordinary skill in the art based upon the present disclosure and can include one or more computer program products. The computer program products can be stored on a non-transitory computer readable medium such as a server memory, a computer network, the Internet, and/or a computer storage device.

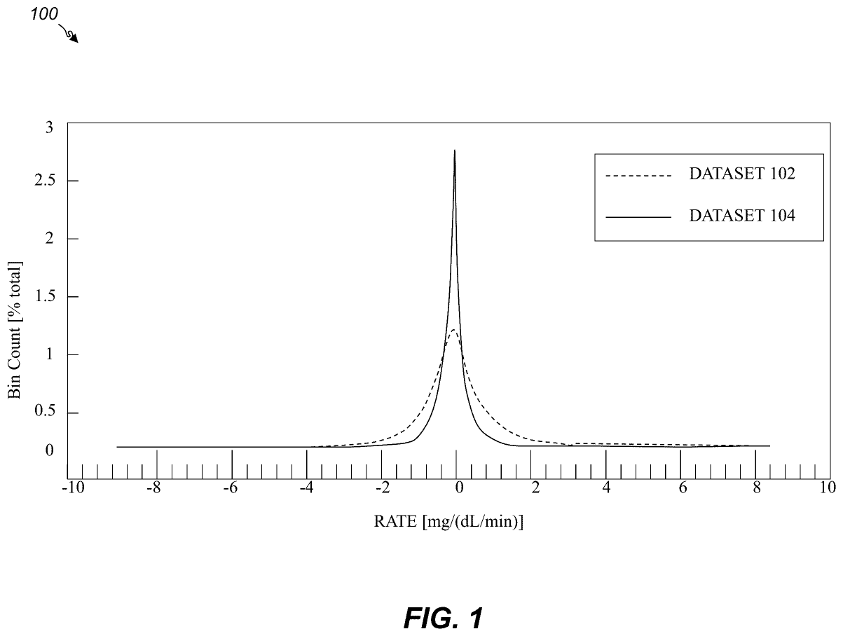

Turning now to FIG. 1, two glucose rates of change distributions from two sensor data datasets are plotted in graph 100. The glucose rates of change are computed from sensor data (e.g. from an analyte measurement system) over all available points in a dataset. Smoothing between values can be performed to improve distribution uniformity. Dataset 102 is taken from measurements of patients with diabetes (PwD) and dataset 104 is taken from measurements of patients without diabetes (PwoD). As can be expected, the glucose rates of change of the PwoD are more concentrated in the middle area, corresponding to a slow/no rate of change, as compared to the distribution of the PwD data. Note that the present invention uses a relatively large number of glucose values (e.g., sensor data) in order to obtain a useful rate distribution metric. In the case of self-monitored blood glucose measurement via an in vitro glucose meter, this may mean taking frequent enough finger stick values over the course of many hours. In the case of an in vivo analyte monitoring system that collects sensor data (such as a CGM or other type of sensor glucose monitor), a significantly shorter data collection duration can suffice.

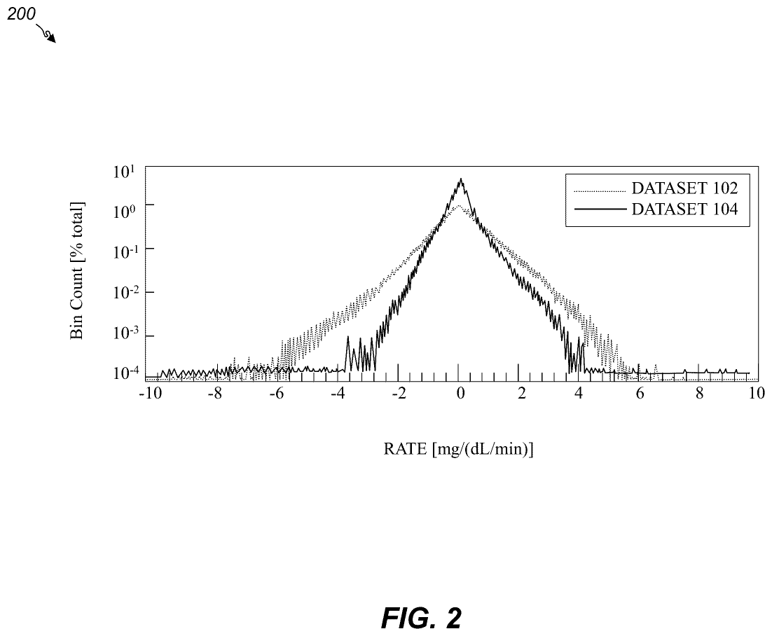

FIG. 2 depicts glucose rate of change distribution from the same datasets 102, 104 shown in FIG. 1, with the distribution count (on the y-axis) shown on a logarithmic scale in graph 200. Note that the distinction in glucose variability between PwD and PwoD can be more clearly discerned over a wider range of rates of change. Unlike FIG. 1, the transformed distribution is shaped such that a simple linear fit could be performed on each direction of the rates of change. The slope of this best fit line reflects the tightness of the distribution of the rates of change. The steeper the absolute slope, the tighter the distribution.

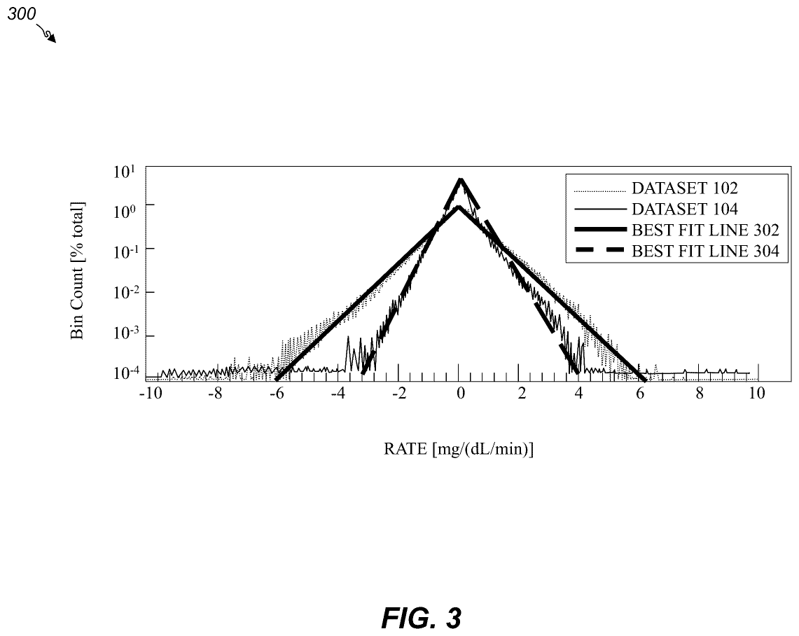

FIG. 3 illustrates the same transformed distributions as FIG. 2 but with a straight thick solid line representing the best-fit line 302 for the PwD rate distribution and a straight thick dashed line representing the best-fit line 304 for the PwoD rate distribution in graph 300. The slope of the best-fit line 304 taken from the transformed PwoD rate distribution dataset 104 is much steeper than that of the best-fit line 302 corresponding to the transformed PwD rate distribution dataset 102. Similarly, patients with diabetes who maintain a better glycemic control level will have best-fit lines with steeper slopes compared to patients with diabetes with a poorer glycemic control level.

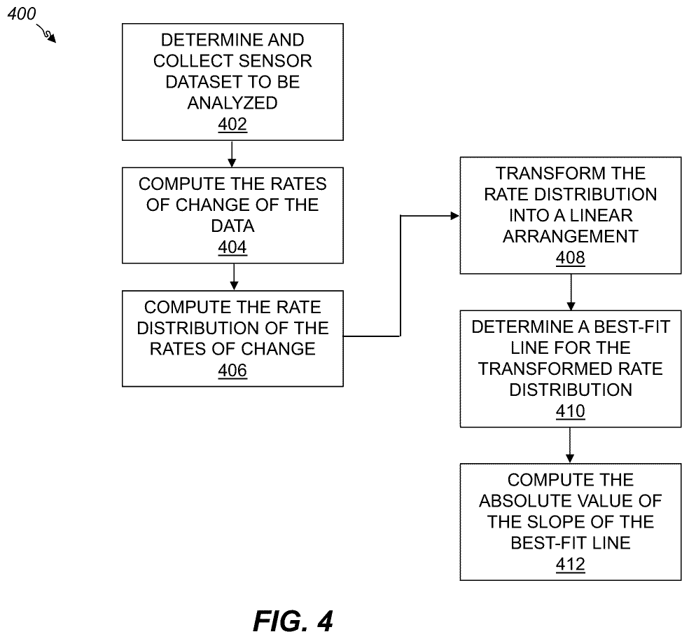

Turning now to FIG. 4, a flowchart depicting an example method 400 according to embodiments of the present invention is provided. Sensor data is collected using an analyte measurement system (e.g., a continuous glucose monitor) (402). In some embodiments, the sensor data is calibrated and/or scaled into glucose concentration units. Note that the method 400 can be applied to sensor data that is currently being received from an analyte measurement system (e.g., a real-time application) and/or to stored sensor data that was previously received (e.g., a retrospective application). For a real-time implementation, sensor data is collected within a moving time window of a fixed period starting at a point in the past up to the present time. For a retrospective implementation, stored sensor data is used in a moving time window of a fixed period starting at a point in the past up to a future point in time.

Once the dataset is defined, the rates of change of the data are computed (404). In other words, for each analyte level measurement, relative to a prior measurement, the amount of change in the analyte concentration level per unit time is computed. Next, based on the computed rates of change of the data, the rate distribution of the rates of change are computed (406). In some embodiments, the distribution of the rates of change are being plotted as shown in FIG. 1 described above. The y-axis of the distribution of the rates of change can then be transformed into a logarithmic scale (408) as shown in FIG. 2 described above. In some embodiments, different scales/transforms are used. For example, instead of a logarithmic scale, a power scale, a square-root scale, or other scale is used to transform the plot of the distribution to taper off from zero rate in a linear fashion. The example in FIG. 2 uses a base-ten, logarithmic transformation. Other base values can also be used. Once a transformation that renders the distribution in a linear manner has been found and computed, a best-fit line is determined, e.g., for both positive and negative rate sides of the transformed distribution (410). For example, the best-fit line can be determined using a common “least-squares error” fit method, an orthogonal fit method, a method of averages, or other well-known methods. Examples of best-fit lines for the positive and negative rates are illustrated in FIG. 3. In some embodiments, the absolute value of the slopes of the positive and negative rate sides of the transformed distribution are then calculated (412). These values represent a simple objective measure of the variability of the analyte concentration and can be used in various applications as mentioned above and described in more detail below.

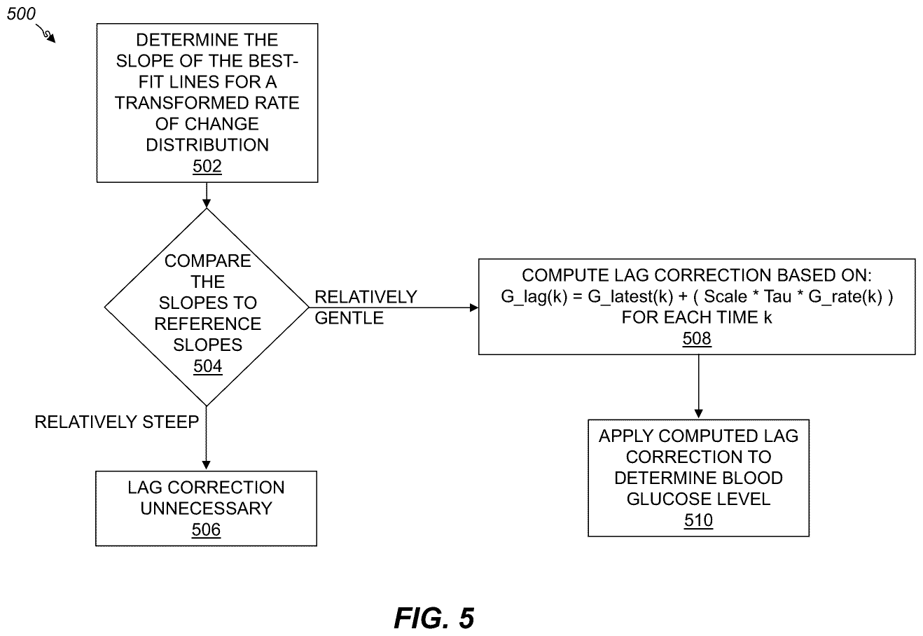

When applied to lag correction of glucose using analyte monitoring system (e.g., CGM) sensor data measuring glucose in interstitial fluid, the degree of glycemic variability can be used to determine the relative benefit of relying on the computed glucose rate of change for lag correction versus the risk of reduced precision caused by amplifying noise and other artifacts. The method 500 of determining how much lag correction to apply is described with reference to the flowchart of FIG. 5. Using the method 400 of FIG. 4 described above, the absolute value of the slopes of the positive and negative rate sides of a transformed rate of change distribution are determined (502). The slopes are compared to one or more reference slopes (504). A predetermined reference slope can be used. The units of this slope are arbitrary and are influenced by the choice of the transformation function. For example, using a logarithmic transformation function, the base can be tuned such that the absolute value of the reference slope equals a convenient integer, such as 2. Other values for a predetermined reference slope can be used. In some embodiments, the slopes can additionally or alternatively be compared to the slopes of sensor data collected from prior time periods.

If the latest slope is relatively steep, then the glucose variability is relatively low. In this case, lag correction is relatively unnecessary (506). Conversely, if the latest slope is gentle (i.e., not steep) compared to the reference, lag correction becomes relatively more important and the method proceeds to compute a correction (508). Depending on a separately determined noise metric, the amount of lag correction applied can vary from 0 to 100%. The noise metric is directly related to the variability of the rate of change calculation, G_rate. If G_rate is calculated from an average of first differences of glucose values in a predetermined window of time, say for example, 15 minutes, then one noise metric can be calculated by taking the standard deviation of the first difference values in that window. For example, in some embodiments, the amount of lag correction to apply is determined (508) based upon the following equation:

$$G\_lag ( k ) = G\_latest ( k ) + ( K * τ * G\_rate ( k ) ) \qquad ( Equation 1 )$$

where G_latest(k) represents the latest interstitial glucose estimate at time k, K represents a scaling factor that determines the amount of lag correction necessary, varying from 0 to 1. The scale K is determined based on two components: a comparison of the computed slope against a reference slope (504) and the noise metric. For example, suppose the slope comparison generates a ratio Rs, and the noise metric generates a ratio N. The slope comparison ratio Rs approaches zero for gentle slopes, and approaches one for steep slopes. The noise metric N approaches one as the sensor signal becomes noisier, and approaches zero otherwise. Then, the scale K can be computed as a product of Rs and N. Alternatively, the scale K can be computed as the smaller of Rs or N. Tau (τ) represents the assumed time constant of lag correction, computed a priori based on population data, and G_rate(k) represents the computed glucose rate of change at time k. Thus, for an unchanging noise characteristic, a relatively steep glucose rate of change distribution slope results in a lower value of scale K. A relatively gentle glucose rate of change distribution slope results in a higher value of scale K. When glucose levels are not changing by a significant amount due to relatively good glycemic control, the risk of reducing precision (i.e., increasing noise) may outweigh the benefit of increasing accuracy (i.e., reducing lag) in the process of lag correcting in the presence of a certain level of signal noise. The calculated lag correction for each time k is applied to the measured interstitial fluid glucose level to more accurately represent the patient's blood glucose level at each time k (510).

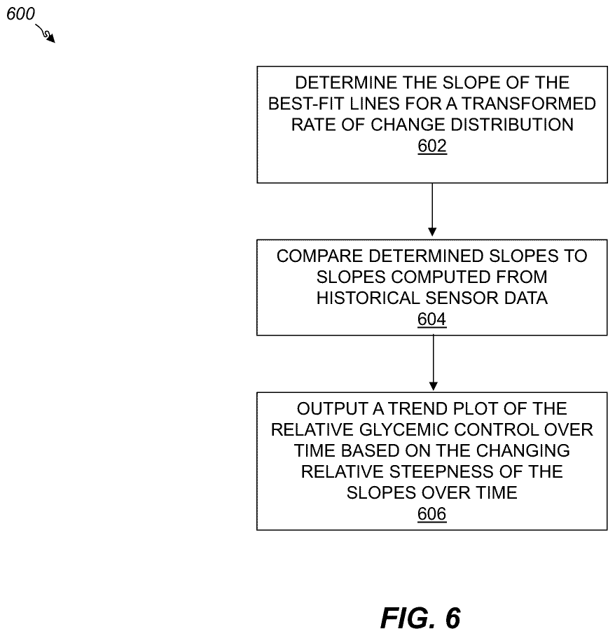

In other embodiments, the degree of glycemic variability is used to assess glycemic control for diabetes treatment evaluation, treatment adjustment, or other purposes. For example, a method 600 of monitoring glycemic control is implemented as depicted in the flowchart of FIG. 6. Using the method 400 of FIG. 4 described above, the absolute value of the slopes of the positive and negative rate sides of a transformed rate of change distribution are determined (602). In some embodiments, the slopes are then compared to a record of slopes computed from historic sensor data stored from prior uses of an analyte monitoring system (604). For example, a database that stores transformed plots of rate of change distributions and corresponding best-fit lines for different “wears” of an analyte monitoring system sensor can be used to determine the relative steepness and thus, the relative amount of glycemic control of the patient compared to their past performance. A trend plot of relative glycemic control over time can be graphed and output by the system (606).

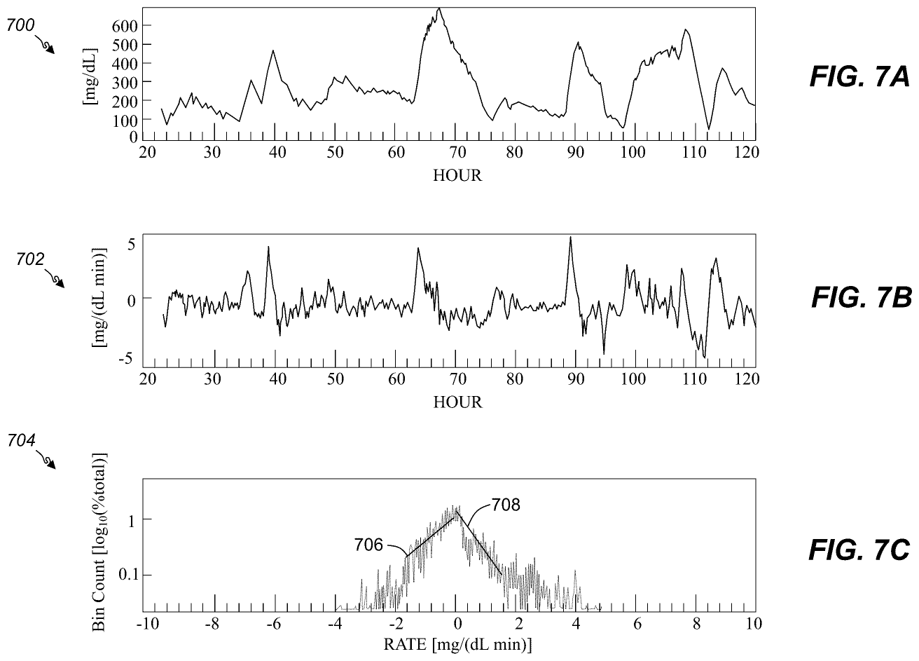

Turning to FIGS. 7A to 7C, graphs 700, 702, 704 are provided representing example data collected from a patient with relatively poor glycemic control. A glucose level plot 700 over time in FIG. 7A shows a relatively high mean glucose level and indicates that a significant amount of time is spent with the glucose level changing in value. The rate of change plot 702 in FIG. 7B confirms this given the significant variance from the zero line. The transformed plot 704 of the distribution of the rate of change in FIG. 7C further confirms this observation as reflected by the slopes of the best fit lines 706, 708.

The positive rate slope 708 is steeper than the negative rate slope 706, as also indicated by the relatively faster glucose level increases compared to the decrease towards lower glucose levels. In some embodiments, the relative steepness of the positive and negative rate distributions can also be used to refine the patient's treatment regimen. For example, by adjusting the lead-time between pre-prandial bolus and actual meals, the glucose level increase can be tempered down. In addition, by changing the timing and amount of correction bolus to allow for a faster initial postprandial glucose recovery followed by a smaller correction bolus later on, a softer “landing” towards normoglycemia can be achieved.

In addition to using glycemic variability to inform the decision whether to apply lag correction, the glycemic range can also be useful in avoiding amplifying noise and artifacts in the sensor data. As mentioned above, at the low glucose range, the presence of signal artifacts such as dropouts significantly impact real-time lag correction of glucose levels measured by the analyte monitoring system. As a patient's level of glycemic control varies over time, their glucose range (i.e., max, min, median glucose levels) varies. When glycemic control is relatively good, the ratio between rate calculation error and true rate is typically larger than when glycemic control is relatively poor. Thus, according to the present invention, the extent of lag correction is scaled back during critical conditions (e.g., such as the patient's glucose level being in the low range), by using historical glucose levels to determine the likelihood of conditions that warrant scaling back of lag correction.

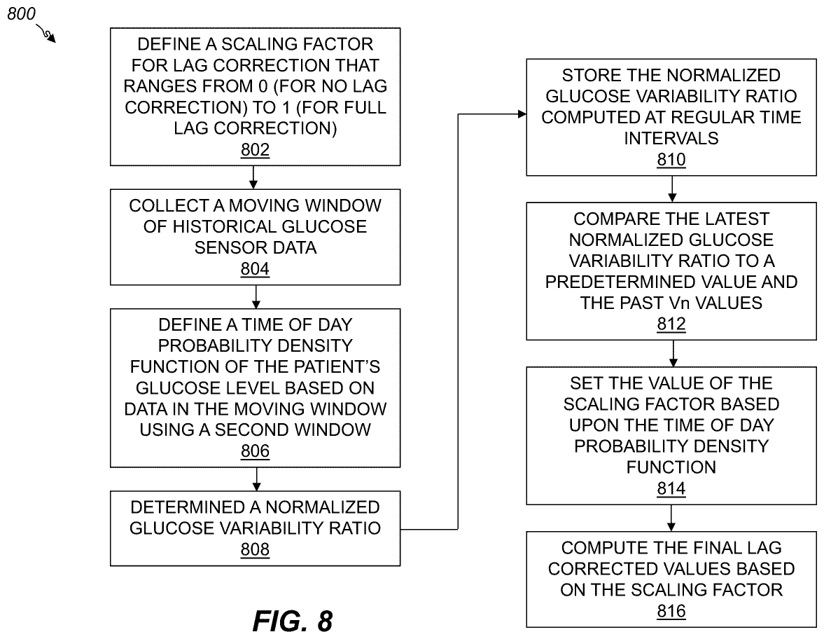

Turning now to FIG. 8, a method 800 for determining an amount of lag correction to apply to sensor data from glucose measurement of interstitial fluid based on glucose level range is depicted in a flowchart. A scaling factor K is defined for lag correction that takes the value from 0 (for no lag correction) to 1 (for full lag correction) (802). For example, let a non-lag corrected glucose value at any time t be G_latest(t), the nominal lag correction amount be G_c(t), and the final lag corrected value be G_lag(t), such that:

$$G\_lag ( t ) = G\_latest ( t ) + ( KG\_c ( t ) ) \qquad ( Equation 2 )$$

A moving window of historical glucose sensor data is collected (804). The period of sensor data collection can be on the order of two to three days. In some embodiments, the data includes sensor data from prior sensor wears from the same patient. A time of day probability density function p(tod) of the patient's glucose level based on data in the moving window is defined using a second window size, for example, on the order of two to three hours (806). A normalized glucose variability ratio, Vn(t) is determined (808). An example of a normalized glucose variability ratio is the ratio of glucose standard deviation to glucose mean within the moving window (or other similar metric) that computes variability normalized to the overall value. Other examples of variability aside from standard deviation include the absolute distance between the upper and lower quartile of the glucose level in the moving window. An additional example includes the absolute distance between the median glucose and a percentile (e.g., the tenth percentile) of the glucose in the window. Examples of an overall value aside from mean glucose include the median glucose, the average of a middle range (e.g., the 45^{th} and 55^{th} percentile) glucose values in the window, etc. The normalized glucose variability ratio Vn(t) computed at regular intervals is stored (810). In some embodiments, the regular intervals are on the order of every 2 to 3 days, for example. The latest normalized glucose variability ratio Vn(t) is compared to a predetermined value Vo and the past Vn values (812). Vo is computed a priori from population data.

The value of the scaling factor K is set based upon the time of day probability density function p(tod) (814). At a time of day when the time of day probability density function p(tod) predicts a high probability of low average glucose, or when the variability from the historic window is very low, K is set close to 0. Otherwise, K is set close to 1. For example, the p(tod) can be used to determine the probability of glucose being lower than, e.g., 100 mg/dL (within a 2 to 3 hour window at the current time of day). This probability can be defined as pLow(tod), which takes on the value of 1 when the probability is 100%, and 0 when the probability is 0%. Then, the scaling factor for lag correction can be computed at any time (and given that time of day) using the equation:

$$K ( t , tod ) = min ( kLow , kN Var , kR Var ) \qquad ( Equation 3 )$$

where kLow represents the gain that mitigates against historic glucose-based, predicted low glucose (kLow=1−pLow(tod)), kNVar represents the gain that mitigates against Vo normalized glucose variability (kNVar=Vn(t)/Vo(t)), and kRVar represents the gain that mitigates against past Vn normalized glucose variability (kRVar=Vn(t)/max([Vn(t−N), Vn(t−N+1), . . . , Vn(t−2), Vn(t−1)])). In this example, N can be on the order of 1 week. Hence, K(t,tod) is the smallest of the three values, kLow, kNVar, kRVar, computed at any time t. The final lag corrected values are computed using Equation 2 based on the scaling factor computed in Equation 3 (816).

Various other modifications and alterations in the structure and method of operation of the embodiments of the present disclosure will be apparent to those skilled in the art without departing from the scope and spirit of the present disclosure. Although the present disclosure has been described in connection with certain embodiments, it should be understood that the present disclosure as claimed should not be unduly limited to such embodiments. It is intended that the following claims define the scope of the present disclosure and that structures and methods within the scope of these claims and their equivalents be covered thereby. Furthermore, the disclosure herein may use terms “define” or “defining” interchangeably with the terms “determine” and “determining.”

What is claimed is:

1. A system for monitoring glucose, comprising:
a glucose sensor, wherein at least a portion of the glucose sensor is configured to be in fluid contact with a fluid below a skin surface of a subject;
one or more processors; and
memory on which one or more instructions are stored that, when executed by the one or more processors, cause the one or more processors to perform operations comprising:
receiving sensor data from the glucose sensor, the sensor data being associated with a time window;
determining a rate of change distribution of the sensor data;
determining a first slope that corresponds to a line of the rate of change distribution;
comparing the first slope with a first reference slope;
generating, based on comparing the first slope with the first reference slope, an output; and
transmitting the output to a display or a transmitter.

2. The system of claim 1, wherein the output represents a trend of a glucose level of the subject.

3. The system of claim 2, wherein the output is a trend plot.

4. The system of claim 1, wherein the first reference slope is based on a historical sensor data.

5. The system of claim 4, wherein the historical sensor data is associated with a time period that is prior to the time window.

6. The system of claim 1, wherein the first slope represents a best-fit line of the rate of change distribution.

7. The system of claim 1, wherein the operations further comprise:
determining a second slope that corresponds to a second line of the rate of change distribution.

8. The system of claim 7, wherein the operations further comprise:
comparing the second slope to a second reference slope; and
generating, based on comparing the second slope with the second reference slope, a second output.

9. The system of claim 7, wherein the first slope corresponds to a positive rate slope and the second slope corresponds to a negative rate slope.

10. The system of claim 9, wherein the operations further comprise:
comparing the first slope with the second slope; and
generating, based on comparing the first slope and the second slope, a modification of a treatment regimen for the subject.

11. A method, by one or more processors associated with a glucose sensor, wherein at least a portion of the glucose sensor is configured to be in fluid contact with a fluid below a skin surface of a subject, the method comprising:
receiving sensor data, the sensor data being associated with a time window;
determining a rate of change distribution of the sensor data;
determining a first slope that corresponds to a line of the rate of change distribution;
comparing the first slope with a first reference slope;
generating, based on comparing the first slope with the first reference slope, an output; and
transmitting the output to a display or a transmitter.

12. The method of claim 11, wherein the output represents a trend of a glucose level of the subject.

13. The method of claim 12, wherein the output is a trend plot.

14. The method of claim 11, wherein the first reference slope is based on a historical sensor data.

15. The method of claim 14, wherein the historical sensor data is associated with a time period that is prior to the time window.

16. The method of claim 11, wherein the first slope represents a best-fit line of the rate of change distribution.

17. The method of claim 11, further comprising:
determining a second slope that corresponds to a second line of the rate of change distribution.

18. The method of claim 17, further comprising:
comparing the second slope to a second reference slope; and
generating, based on comparing the second slope with the second reference slope, a second output.

19. The method of claim 17, wherein the first slope corresponds to a positive rate slope and the second slope corresponds to a negative rate slope.

20. The method of claim 19, further comprising:
comparing the first slope with the second slope; and
generating, based on comparing the first slope and the second slope, a modification of a treatment regimen for the subject.
